(19) United States
(12) Patent Application Publication
(10) Pub. No.: US 2007/0036744 A1
(43) Pub. Date: Feb. 15, 2007

(54) Treatment or prevention of respiratory viral infections with alpha thymosin peptides

(75) Inventors: Alfred R. Rudolph, Los Altos Hills, CA (US); Cynthia W. Tuthill, Menlo Park, CA (US)

Correspondence Address: ROTHWELL, FIGG, ERNST & MANBECK, P.C., 1425 K STREET, N.W., SUITE 800, WASHINGTON, DC, 20005, US

(73) Assignee: SciClone Pharmaceuticals, Inc., San Mateo, CA (US)

(21) Appl. No.: 10/553,317

(22) PCT Filed: Apr. 23, 2004
(86) PCT No.: PCT/US04/12663

§ 371 Date: Sep. 26, 2006

Related U.S. Application Data
(60) Provisional application No. 60/464,645, filed on Apr. 23, 2003.
(60) Provisional application No. 60/470,420, filed on May 15, 2003.

(51) Int. Cl. A61K 38/21; A61K 38/17

(52) U.S. Cl. 424/78.27; 514/12; 424/85.7

(57) ABSTRACT

An alpha thymosin peptide is administered to a patient having, or at risk of a respiratory viral infection, coronavirus infection and/or SARS.

### CROSS-REFERENCE TO RELATED APPLICATIONS

The present application claims benefit of U.S. Provisional Application Ser. No. 60/464,645, filed Apr. 23, 2003, and U.S. Provisional Application Ser. No. 60/470,420, filed May 15, 2003.

### FIELD OF THE INVENTION

The present invention relates to the field of treatment of respiratory viral infections.

### DESCRIPTION OF THE BACKGROUND ART

Severe acute respiratory syndrome (SARS) is a viral respiratory illness caused by a coronavirus, called SARS-associated coronavirus (SARS-CoV). SARS was first reported in Asia in February 2003. Over the next few months, the illness spread to more than two dozen countries in North America, South America, Europe, and Asia.

In general, SARS begins with a high fever (temperature greater than 100.4° F. [>38.0° C.]). Other symptoms may include headache, an overall feeling of discomfort, and body aches. Some people also have mild respiratory symptoms at the outset. About 10 percent to 20 percent of patients have diarrhea. After 2 to 7 days, SARS patients may develop a dry cough. Most patients develop pneumonia.

The main way that SARS seems to spread is by close person-to-person contact. The virus that causes SARS is thought to be transmitted most readily by respiratory droplets (droplet spread) produced when an infected person coughs or sneezes. Droplet spread can happen when droplets from the cough or sneeze of an infected person are propelled a short distance (generally up to 3 feet) through the air and deposited on the mucous membranes of the mouth, nose, or eyes of persons who are nearby. The virus also can spread when a person touches a surface or object contaminated with infectious droplets and then touches his or her mouth, nose, or eye(s). In addition, it is possible that the SARS virus might spread more broadly through the air (airborne spread) or by other ways that are not now known.

According to the World Health Organization (WHO), a total of 8,098 people worldwide became sick with SARS during the 2003 outbreak. Of these, 774 died.

There remains a need in the art for the treatment or prevention of respiratory viral infections such as SARS.

### SUMMARY OF THE INVENTION

In accordance with the present invention, a method of treatment or prevention of a respiratory viral infection in a patient comprises administering to the patient an effective amount of an alpha thymosin peptide.

### DETAILED DESCRIPTION OF THE INVENTION

In accordance with one embodiment, the present invention relates to treatment or prevention of respiratory viral infections by administering an alpha thymosin peptide to a patient.

In accordance with another embodiment, the invention relates to treatment or prevention of coronavirus infection by administering an alpha thymosin peptide to a patient.

In accordance with a further embodiment, the invention relates to treatment or prevention of Severe Acute Respiratory Syndrome (SARS) in a patient by administering an alpha thymosin peptide.

Administration for prevention can be to persons at high risk because of contact with suspected disease carriers, or in carriers who are asymptomatic.

Alpha thymosin peptides comprise thymosin alpha 1 (TA1) peptides including naturally occurring TA1 as well as synthetic TA1 and recombinant TA1 having the amino acid sequence of naturally occurring TA1, amino acid sequences substantially similar thereto, or an abbreviated sequence form thereof, and their biologically active analogs having substituted, deleted, elongated, replaced, or otherwise modified sequences which possess bioactivity substantially similar to that of TA1, e.g., a TA1 derived peptide having sufficient amino acid homology with TA1 such that it functions in substantially the same way with substantially the same activity as TA1.

Administration can be by any suitable method, including injection, periodic infusion, continuous infusion, and the like. Suitable dosages of the alpha thymosin peptide can be in the range of about 0.001-10 mg/kg/day.

According to one aspect of this embodiment of the present invention, the dosage unit comprising an alpha thymosin peptide is administered to the patient on a routine basis. For example, the dosage unit can be administered more than once daily, once daily, weekly, monthly, etc. The dosage unit may be administered on a bi-weekly basis, i.e., twice a week, for example, on Tuesday and Saturday. The dosage unit of TA1 may be administered on a thrice weekly basis, i.e., three times per week.

Because the plasma half-life of subcutaneously injected TA1 is only about two hours, according to one embodiment, a TA1 peptide such as TA1 is administered to a patient in need of immune stimulation so as to substantially continuously maintain an immune stimulating-effective amount of the TA1 peptide in the patient's circulatory system during a substantially longer treatment or prevention period. Although much longer treatment periods are contemplated in accordance with the present invention, embodiments of the invention include substantially continuously maintaining an immune stimulating-effective amount of the TA1 peptide in the patient's circulatory system during treatment periods of at least about 6, 10, 12 hours, or longer. In other embodiments, treatment periods are for at least about a day, and even for a plurality of days, e.g., a week or longer. However, it is contemplated that treatments, as defined above, in which immune stimulating-effective amounts of the TA1 peptide are substantially continuously maintained in the patient's circulatory system, may be separated by non-treatment periods of similar or different durations.

In accordance with one embodiment, the TA1 peptide is continuously infused into a patient, e.g., by intravenous infusion, during the treatment period, so as to substantially continuously maintain an immune stimulating-effective amount of the TA1 peptide in the patient's circulatory system. The infusion may be carried out by any suitable means, such as by minipump.

Alternatively, an injection regimen of the TA1 peptide can be maintained so as to substantially continuously maintain an immune stimulating-effective amount of the TA1 peptide in the patient's circulatory system. Suitable injection regimens may include an injection every 1, 2, 4, 6, etc. hours, so as to substantially continuously maintain the immune stimulating-effective amount of the Thymosin alpha 1 peptide in the patient's circulatory system during the treatment period.

Although it is contemplated that during continuous infusion of the TA1 peptide, administration will be for a substantially longer duration, according to one embodiment the continuous infusion of the TA1 peptide is for a treatment period of at least about 1 hour. More preferably, continuous infusion is carried out for longer periods, such as for periods of at least about 6, 8, 10, 12 hours, or longer. In other embodiments, continuous infusion is for at least about one day, and even for a plurality of days such as for one week or more.

In preferred embodiments, the TA1 peptide is present in a pharmaceutically acceptable liquid carrier, such as water for injection, saline in physiological concentrations, or similar.

The present invention also comprises administration of a physiologically active conjugate comprising a TA1 peptide conjugated to a material which increases half-life of the TA1 peptide in serum of a patient when said conjugate is administered to a patient. The material may be a substantially non-antigenic polymer. Suitable polymers will have a molecular weight within a range of about 200-300,000, preferably within a range of about 1,000-100,000, more preferably within a range of about 5,000-35,000, and most preferably within a range of about 10,000-30,000, with a molecular weight of about 20,000 being particularly preferred.

The polymeric substances included are also preferably water-soluble at room temperature. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Among the substantially non-antigenic polymers, mono-activated, alkyl-terminated polyalkylene oxides (PAO's), such as monomethyl-terminated polyethylene glycols (mPEG's) are contemplated. In addition to mPEG, C1-4 alkyl-terminated polymers may also be useful.

As an alternative to PAO-based polymers, effectively non-antigenic materials such as dextran, polyvinyl pyrrolidones, polyacrylamides, polyvinyl alcohols, carbohydrate-based polymers and the like can be used. Those of ordinary skill in the art will realize that the foregoing list is merely illustrative and that all polymer materials having the qualities described herein are contemplated. For purposes of the present invention, “effectively non-antigenic” means all materials understood in the art as being nontoxic and not eliciting an appreciable immunogenic response in mammals.

The polymer may be straight-chain or branched. Polyethylene glycol (PEG) is a particularly preferred polymer.

The polymer can be conjugated to the TA1 peptide by any suitable method. Exemplary methods for conjugating polymers to peptides are disclosed in U.S. Pat. Nos. 4,179,337, 4,766,106, 4,917,888, 5,122,614 and 6,177,074, as well as PCT International Publication No. WO 95/13090, all of which are incorporated herein by reference. Thymosin alpha 1 has five separate possible sites for amino group conjugation of a polymer, and polymer(s) can be conjugated at one or a plurality of sites. According to one embodiment, 20,000 molecular weight PEG is conjugated to the N-terminal end of TA1 to form a PEG-TA1. This can be formed by solid phase peptide synthesis of TA1 on insoluble polymeric support beads, as is known in the art, with appropriate side chain protective groups. After complete synthesis of the TA1 peptide on the beads, the protected TA1 is cleaved from the beads leaving the N-terminus with a free amino group, which is reacted with 20,000 molecular weight PEG. The side chain protective groups then are removed to form a conjugate in accordance with this embodiment of the invention.

The isolation, characterization and use of TA1 peptides is described, for example, in U.S. Pat. No. 4,079,127, U.S. Pat. No. 4,353,821, U.S. Pat. No. 4,148,788 and U.S. Pat. No. 4,116,951. Effective amounts of TA1 peptide can be determined by routine dose-titration experiments. TA1 has been found to be safe for humans when administered in doses as high as 16 mg/kg body weight/day. Preferred dosages of TA1 peptide are within the range of 0.001 mg/kg body weight/day to 10 mg/kg body weight/day. According to one embodiment, immune stimulating-effective amounts are at dosages which include the TA1 peptide in an amount within a range of about 0.1-20 mg. Preferred dosages include the TA1 peptide in an amount within the range of about 0.5-10 mg, more preferably about 1-5 mg, most preferably about 1.6-3.2 mg. The above dosages reflect only the TA1 peptide present in the composition, and not the weight of the polymer, if any, conjugated thereto.

Conjugation of a polymer to a TA1 peptide in accordance with the present invention substantially increases the plasma half-life of the peptide.

The TA1 peptide also can be administered with an effective amount of an interferon, such as interferon alpha, wherein interferon alpha-2b is preferred. Suitable dosages of interferon alpha-2b may be in the range of about 1-3 MU.

The TA1 peptide also can be administered with other immune stimulators or antiviral agents.

1. A method of treatment or prevention of a respiratory viral infection in a patient comprising administering to said patient an effective amount of an alpha thymosin peptide.

2. The method of claim 1 wherein the respiratory viral infection is a result of coronavirus infection.

3. The method of claim 1 wherein said respiratory viral infection is SARS.

4. The method of claim 1 wherein said amount of alpha thymosin peptide is within a range of about 0.1-20 mg.

5. The method of claim 4 wherein said range is about 0.5-10 mg.

6. The method of claim 4 wherein said range is about 1-5 mg.

7. The method of claim 1 wherein said alpha thymosin peptide is thymosin alpha 1.

8. The method of claim 7 wherein said thymosin alpha 1 is administered to said patient at a dosage within a range of about 1-5 mg.

9. The method of claim 8 wherein said dosage is about 1.6-3.2 mg.

10. The method of claim 1, further comprising administering to said patient an effective amount of an interferon.

11. The method of claim 10 wherein said interferon is interferon alpha.

12. The method of claim 11 wherein said amount of said interferon is about 1-3 MU.

13. The method of claim 1 wherein said alpha thymosin peptide is conjugated to a polymer.

14. The method of claim 13 wherein said polymer is polyethylene glycol (PEG).

15. The method of claim 14 wherein said alpha thymosin peptide is PEG-TA1.

16. The method of claim 15 wherein said PEG of said PEG-TA1 has a molecular weight of about 20,000.

17. The method of claim 1 wherein said alpha thymosin peptide is substantially continuously maintained in said patient in an immune stimulating-effective amount.

18. The method of claim 17 wherein said alpha thymosin peptide is administered by continuous infusion into said patient.

19. The method of claim 18 wherein said alpha thymosin peptide is TA1.
